(19) United States
(12) Patent Application Publication
(10) Pub. No.: US 2012/0290102 A1
(43) Pub. Date: Nov. 15, 2012

(54) Knee Crutch System

(76) Inventor: Ormonde M. Mahoney, Athens, GA (US)

(21) Appl. No.: 13/105,647

(22) Filed: May 11, 2011

(51) Int. Cl. A61F 2/78

(52) U.S. Cl. 623/32

(57) ABSTRACT

Disclosed embodiments, as well as features and aspects thereof, are directed towards providing a knee crutch system. Embodiments of a knee crutch system provide support to a user's injured lower leg, safely positioning it such that it bears no weight other than its own, while enabling the user to remain mobile. Moreover, a user of a knee crutch system may enjoy mobility without obligating the use of his arms or hands to stabilize the ambulatory motion or otherwise support body weight.

### CROSS-REFERENCE TO RELATED APPLICATIONS

This application is related to the United States application for design patent filed concurrently herewith under 37 CFR 1.53(d), having a title of KNEE CRUTCH WITH HINGED LEG CRADLE, identified by attorney docket number 13047.1020 and filed under customer number 35856, the entire contents of which are hereby incorporated by reference. This application is also related to the United States application for design patent filed concurrently herewith under 37 CFR 1.53(d), having a title of KNEE CRUTCH, identified by attorney docket number 13047.1030 and filed under customer number 35856, the entire contents of which are hereby incorporated by reference.

### BACKGROUND

Crutches are medical devices designed to aid in mobility by providing support to the body weight of an injured user. When injury or illness makes mobility difficult, or even impossible, the simple solution is often the humble crutch. Injured men have been propping themselves with crutches, of one form or another, for probably as long as man has sustained injuries. Despite eons of use, however, basic crutch designs surprisingly have not changed much.

The first crutches were probably of an underarm design made from forked tree branches. The top of an underarm crutch fits under the arm such that the user can grasp handles located midway down the crutch, thereby enabling the user to lift one or both feet off the ground. In this way, the user can propel forward by placing the crutches in front of his body and then swinging his body forward of the crutches. Continuous use of underarm crutches, however, can be dangerous due to increased possibility of nerve damage in the armpit area. Also, because underarm crutches require the user to grasp handles, a user of underarm crutches may be prevented from using his hands for other purposes.

Therefore, what is needed in the art is a knee crutch system that supports the injured lower leg of a user while allowing the user to remain mobile. Further, what is needed in the art is a knee crutch system that provides mobility to a user without requiring the user to occupy his hands.

### BRIEF SUMMARY

Embodiments of a knee crutch system provide support to a user's injured lower leg, safely positioning it such that it bears no weight other than its own, while enabling the user to remain mobile. Moreover, a user of a knee crutch system may enjoy mobility without obligating the use of his arms or hands to stabilize the ambulatory motion or otherwise support body weight. A user of a given knee crutch embodiment may bend a leg substantially ninety degrees and insert the knee into a cradle component. Once the user's knee is inserted, the cradle component may be configured to substantially fix the user's leg in such position, thereby causing the user's lower leg to be held in a position substantially parallel to the ground when the user's upper leg is in a position substantially perpendicular to the ground. Advantageously, because a given knee crutch system embodiment will include a post component that extends from an exterior surface of the cradle component to the ground, a force attributable to the weight of the user may be translated to the ground via the cradle and post components. That is, a user of a knee crutch system may be able to “stand” and/or “walk” despite the lower leg being positioned at a fixed angle relative to the upper leg.

One embodiment of a knee crutch system that is operable to support the mobility of a user who has an injury below the knee comprises a cradle component, a shaft component and one or more components operable to secure the cradle to a user's leg. The cradle component may be configured substantially in a right angle such that an upper leg portion of the cradle component joins a lower leg portion of the cradle component to define a corner. Thus, the cradle component is particularly well suited to interface to the user by receiving the upper leg of the user into the interior of the upper leg portion, the lower leg of the user into the interior of the lower leg portion and the knee of the user into the interior of the corner. Moreover, the upper leg portion of the cradle partially wraps around the upper leg of the user and the lower leg portion of the cradle is supports the lower leg of the user in a position that is substantially parallel to the ground. The end of the shaft component that is proximate to the cradle is anchored to the cradle component at the exterior of the cradle component's corner. The distal end of the shaft component is configured to interface with the ground and may include a prosthetic foot. The overall position of the shaft may be substantially aligned with the upper leg of the user. The one or more securing components are operable to secure the cradle component to the user.

### BRIEF DESCRIPTION OF THE SEVERAL VIEWS OF THE DRAWING

In the figures, like reference numerals refer to like parts throughout the various views unless otherwise indicated. For reference numerals with letter character designations such as “102A” or “102B”, the letter character designations may differentiate two like parts or elements present in the same figure. Letter character designations for reference numerals may be omitted when it is intended that a reference numeral to encompass all parts having the same reference numeral in all figures.

FIG. 1 is a perspective view of an exemplary knee crutch system having a hinge aspect at the knee of the leg cradle;

FIG. 1A is an enlarged detail view taken at inset circle 1A of the FIG. 1 embodiment;

FIG. 2 is a side elevation view of the FIG. 1 embodiment;

FIG. 3A is a front elevation view of an alternate embodiment of a knee crutch system in a shortened position;

FIG. 3B is a front elevation view of an alternate embodiment of a knee crutch system in an extended position;

FIG. 4 is a perspective view of an exemplary knee crutch system having an integral knee aspect;

FIG. 5 is a top plan view of an exemplary knee crutch system in use by a user; and

FIGS. 6A-6C depict a progression of positions of an exemplary knee crutch system in the act of walking.

### DETAILED DESCRIPTION

The presently disclosed embodiments, as well as features and aspects thereof, are directed towards providing a knee crutch system for supporting the lower leg of a user while providing the user with a means for mobility that does not require the use of his hands.

As will become apparent from a review of the drawings and the following description, a user of a given knee crutch embodiment may bend a leg substantially ninety degrees and insert the knee into a cradle component. Once a user's knee is inserted, the cradle component may be configured to substantially fix the user's leg in such position, thereby causing the user's lower leg to be held in a position substantially parallel to the ground when the user's upper leg is in a position substantially perpendicular to the ground. Advantageously, because a given knee crutch system embodiment will include a post component that extends from an exterior surface of the cradle component to the ground, a force attributable to the weight of the user may be translated to the ground via the cradle and post components. That is, a user of a knee crutch system may be able to “stand” and/or “walk” despite the lower leg being positioned at a fixed angle relative to the upper leg.

Notably, although the various embodiments of a knee crutch system depicted and described in the present disclosure dictate that a user's leg be bent at a substantially ninety degree angle, it is envisioned that other embodiments of a knee crutch system may dictate that a user's leg be bent at an angle that is more obtuse, or acute, than substantially ninety degrees. As such, one of ordinary skill in the art will acknowledge that the particular leg bend angle necessitated by a given knee crutch system embodiment will not limit the scope of the disclosure. Moreover, it is envisioned that some embodiments of a knee crutch system may accommodate multiple leg bend angles according to the preference of the user.

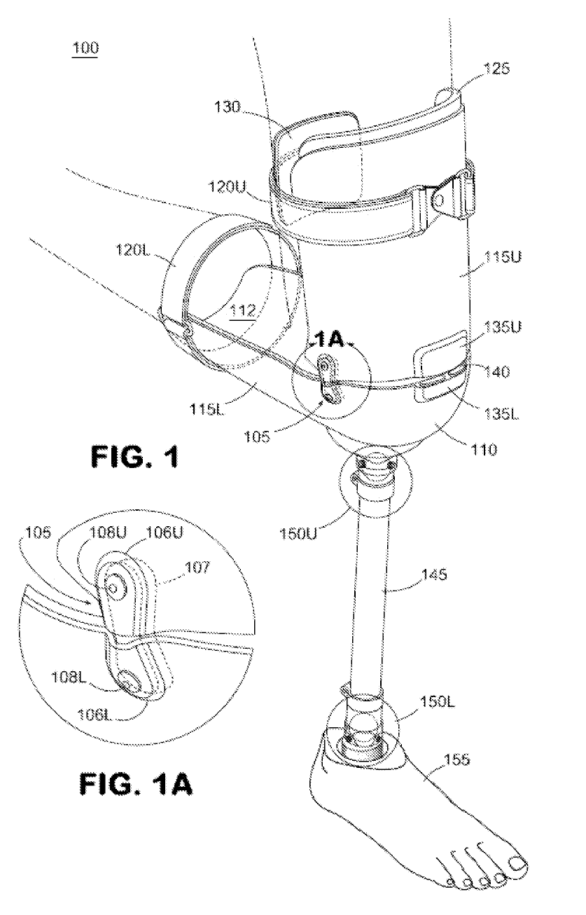

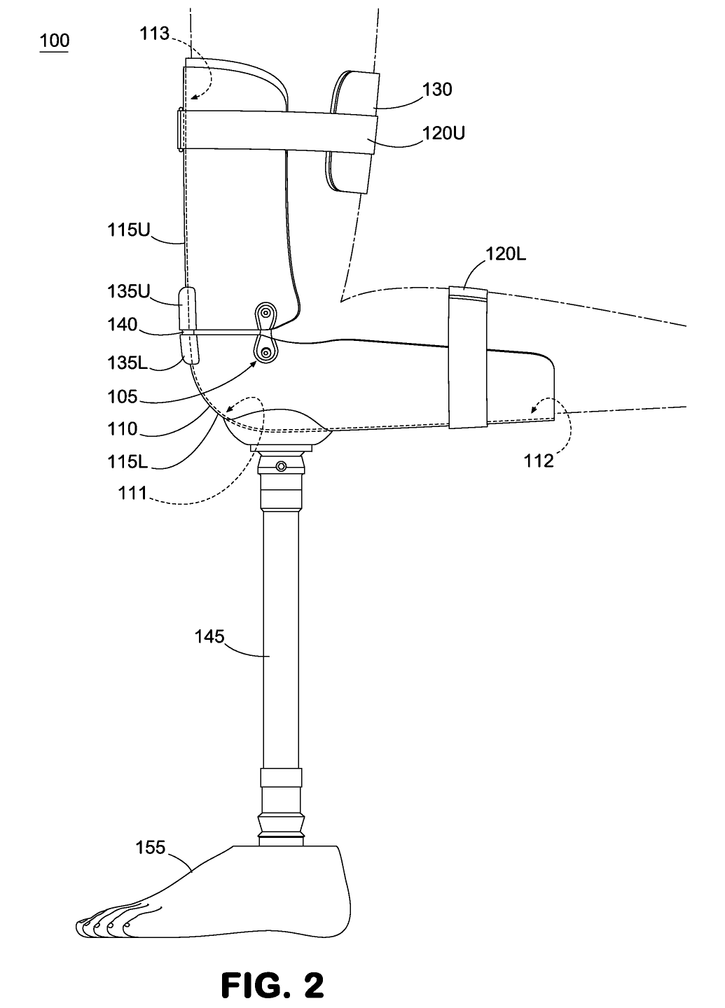

FIG. 1 is a perspective view of an exemplary knee crutch system 100 having a hinge aspect 105 at the knee 110. In the embodiment 100, the cradle component 115 includes an upper leg portion 115U and a lower leg portion 115L. The lower leg portion 115L may be configured to receive the knee and shin of a user such that the user's knee can be inserted into an interior knee cavity 111 (see FIG. 2) that is defined by the knee 110 of the lower leg portion 115L. Similarly, the shin of a user may be received into an interior shin cavity 112 defined by lower leg portion 115U as it extends away from the knee 110. With a user's knee inserted into the interior knee cavity 111 and shin inserted into the interior shin cavity 112, the thigh of the user may be received by an interior thigh cavity 113 (see FIG. 2) defined by the upper leg portion 115U.

Notably, as can be seen in the various exemplary embodiments illustrated in the figures, the upper and lower leg portions 115U, 115L may be configured to extend partially around the circumference of a user's leg. It is an advantage of some embodiments having such upper and lower leg portions 115U, 115L that a user's upper and/or lower leg is provided a measure of protection from extraneous hazards.

Once a user's leg is inserted into the various cavities 111,112,113 of cradle 115 outlined above, the user's upper and lower leg may be secured to the cradle component 115 by way of upper and lower fasteners 120U, 120L. Notably, in the various exemplary embodiments, upper and lower fasteners 120U, 120L are depicted as straps operable to be adjusted in length and secured in place via a “hook and loop” feature, such as VELCRO, as is known in the art. It will be understood, however, that a knee crutch system is not limited to include fastener mechanisms comprised of VELCRO straps and, as such, may include, without limitation, any means for fastening to the user whether such means be adjustable in length or not. For example, it is envisioned that the fasteners 120 may include belts with buckles, elastic straps or the like. Moreover, a knee crutch system is not limited to include only an upper and lower fastener mechanism such as that depicted in the exemplary FIG. 1 embodiment. Rather, it is envisioned that any given embodiment of a knee crutch system may include any number of fastening mechanisms.

Returning to the exemplary FIG. 1 embodiment, upper and lower leg portions 115U, 115L are joined at the knee area 110 via a hinge mechanism 105. Hinge mechanism 105 may collectively include complimentary instances on the left 105L (not shown) and right 105R sides of cradle 115. Briefly referring to the enlarged detail view of FIG. 1A, it can be seen that the exemplary hinge mechanism 105 includes complimentary upper and lower molded sockets 106U, 106L positioned, respectively, on the exterior surfaces of upper and lower leg portions 115U, 115L. The upper and lower molded sockets 106U, 106L are configured to receive the upper and lower ends, respectively, of a flexible grommet component 107. The ends of flexible grommet component 107 are secured in the upper and lower molded sockets 106U, 106L by way of upper and lower mounting screws 108U, 108L. Advantageously, because the flexible grommet component 107 provides the communication between the upper and lower leg portions 115U, 115L, one of ordinary skill in the art will recognize that upper leg portion 115U may be rotationally translated from a first upright position to a second inclined position, relative to lower leg portion 115L, thus providing a user with a range of motion through the user's knee.

While the particular hinge mechanism 105 depicted and described relative to the FIG. 1 embodiment may be novel in, and of, itself, it will be understood that a knee crutch system having a hinged aspect is not limited to include a hinge mechanism such as the one just described. Rather, it is envisioned that any given embodiment of a knee crutch system may or may not include any hinging mechanism that may be known in the art. The presence or absence of a hinging mechanism, or the particular type of hinging mechanism included, will not be a limiting aspect to a knee crutch system embodiment that falls within the scope of the present disclosure.

Further, as can be seen in the FIG. 1 embodiment, a knee crutch system may include padding elements such as padding 125. Notably, while padding is not required in all embodiments, it is envisioned that various padding systems and/or arrangements may be included in any given knee crutch system embodiment for the primary purpose of providing comfort to a user. Similarly, elements such as the exemplary rear thigh plate 130 may be included in some embodiments for the primary purpose of increasing user comfort through the more even distribution of the force provided by the fastening mechanisms 120.

Additionally, although not required in all embodiments, it is envisioned that various mechanisms for cushioning the mating point between the upper and lower leg portions 115U, 115L may be included in a given hinged knee crutch system embodiment. For example, in the FIG. 1 embodiment 100, upper and lower bumper plates 135U, 135L are positioned, respectively, on the lower exterior surface of the upper leg portion 115U and the upper exterior surface of the knee area 110 of lower leg portion 115L. Advantageously, the addition of the bumper plates 135U, 135L in the embodiment 100 provide surfaces for receipt of rubber bumpers 140 that may cushion the upper leg portion 115U as it is translated to a substantially upright position relative to lower leg portion 115L.

From the exterior surface of lower leg portion 115L, post 145 extends toward the ground, thereby providing a means for support of a user's weight. Advantageously, post 145 may be positioned such that it is substantially in line with the thigh of a user. That is, one of ordinary skill in the art will recognize that post 145 may be placed relative to the femur bone (not shown) of a user such that the position of post 145 correlates to the natural standing position of the user's tibia bone (not shown).

Post 145 may be in communication with lower leg portion 115L by way of an upper ball and socket joint 150U. Notably, joint 150 is not required in all embodiments of a knee crutch system and, as such, the inclusion or exclusion of a ball and socket joint 150, or any other type of joint 150, will not limit the scope of the disclosure. Also, it is envisioned that some embodiments of a knee crutch system may further include a lockable hinge mechanism (not shown) located between joint 150 and the base of cradle 115 such that the post 145, when not in use, may be translated to a position that is substantially parallel to the exterior surface of lower leg portion 115L.

Returning to FIG. 1, the upper ball and socket joint 150U may generally include a ball located between two rotator cuffs and settable by a series of dog point screws. One of ordinary skill in the art will recognize that it is an advantage of a knee crutch system embodiment which includes a joint 150, whether such joint be of the ball and socket type or not, that the angle position of post 145 may be adjustable relative to the thigh of the user. Similarly, at the lower end of post 145, some embodiments of a knee crutch system may include a joint 150L. One of ordinary skill in the art will recognize that a joint 150L may serve to provide adjustability of the position of a prosthetic foot 155 relative to post 145.

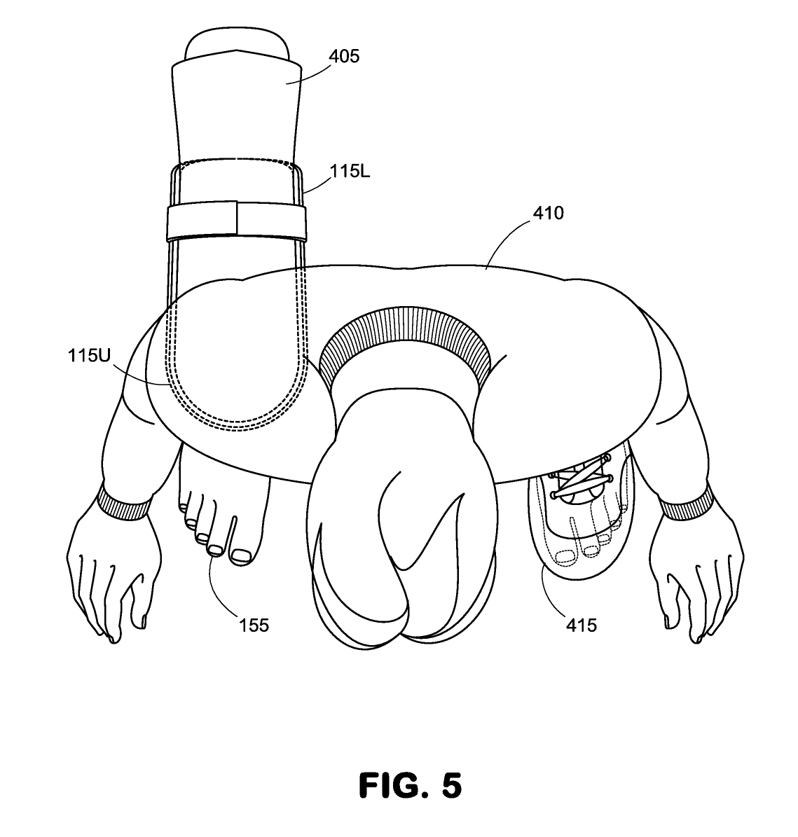

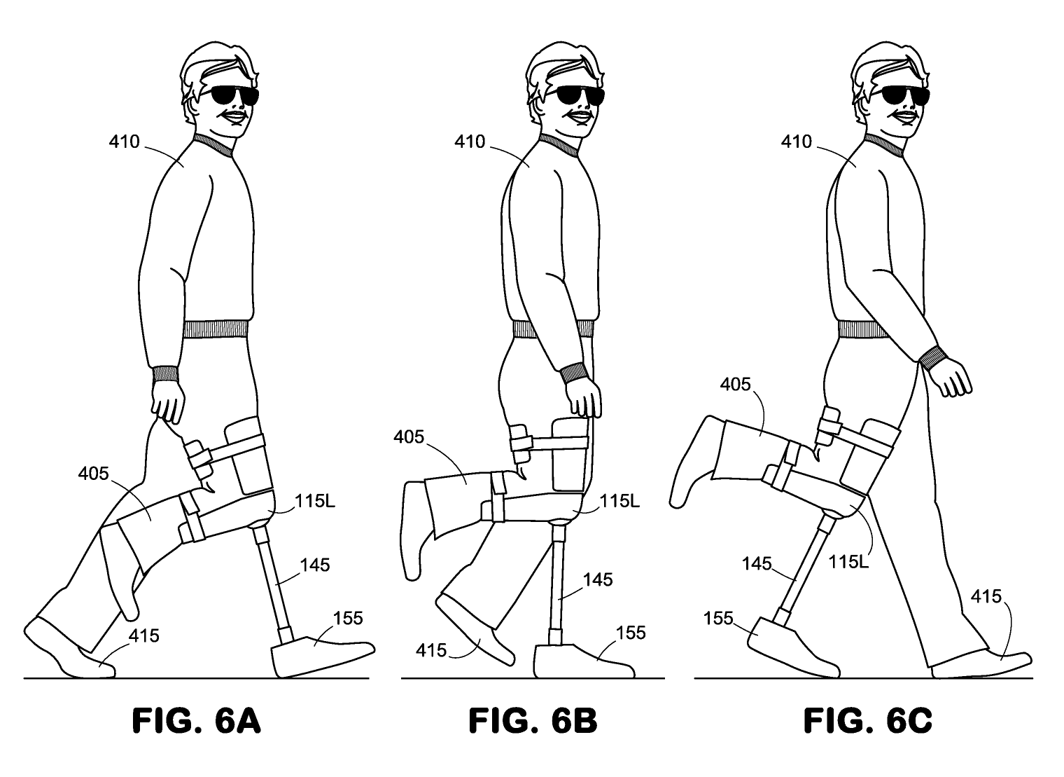

It is an advantage of knee crutch system embodiments, such as the exemplary embodiment 100 depicted in FIG. 1, that a prosthetic foot 155 may be fixedly attached to the distal end of post 145 such that the prosthetic foot 155 is positioned substantially in correlation with the position of a user's natural foot when standing. This advantage, and other advantages, will be described in more detail in connection with FIGS. 5-6.

FIG. 2 is a side elevation view of the knee crutch system embodiment 100 illustrated in FIG. 1. In the FIG. 2 illustration, the leg of a user can be seen inserted into interior knee, shin and thigh cavities 111, 112, 113 as described above. Further, the FIG. 2 illustration depicts the user's leg in a position that is secured to upper and lower leg portions 115U, 115L via upper and lower fasteners 120U, 120L.

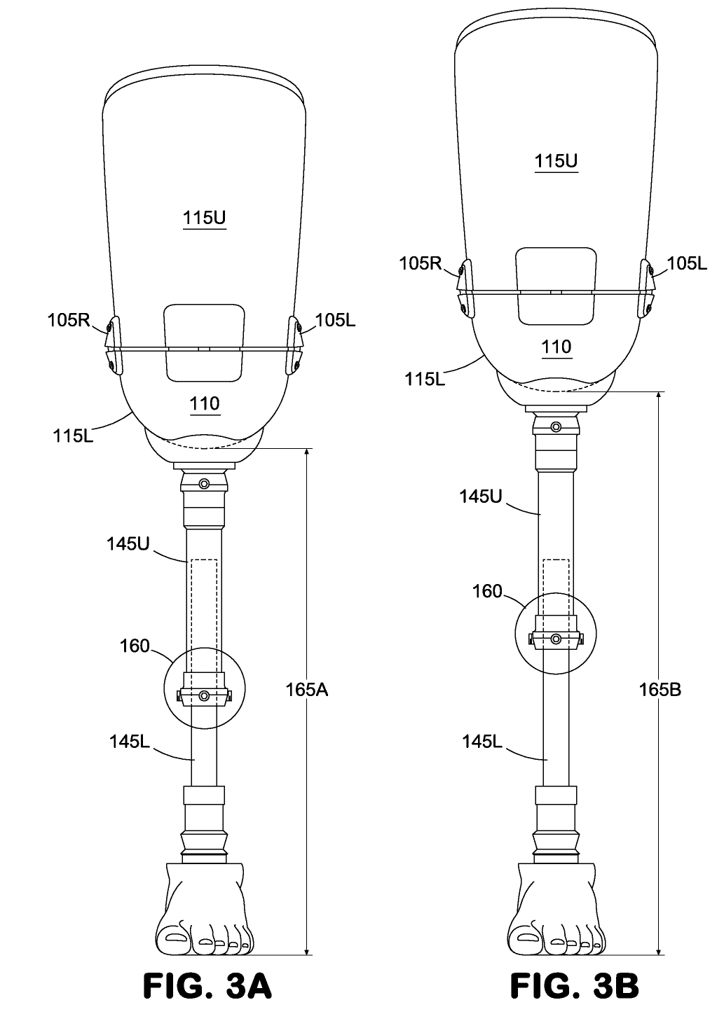

FIGS. 3A-3B are front side elevation views of an exemplary knee crutch system having a height adjustment aspect 160. The FIG. 3 embodiment is substantially the same as that which has been described relative to the FIG. 1 embodiment 100. However, the exemplary embodiment 200 further includes a height adjustment mechanism 160. As can be seen in the FIG. 3 illustration, embodiments may include a lower post 145L configured to be slidably inserted into upper post 145U and fixed in position via a collar 160 with set screws.

Advantageously, by leveraging a post component 145 having an upper portion 145U and a lower portion 145L, a given knee crutch system embodiment may provide a user with a means for adjusting the height 165 of the entire knee crutch system. More specifically, referring to the FIG. 3A illustration, the exemplary knee crutch system may be set at a certain height 165A to accommodate a first user. Subsequently, height 165A may be adjusted to a second height 165B (FIG. 3B) to accommodate a second, taller user.

Notably, the collar 160 with set screws is offered for illustrative purposes only and is not intended to, nor does it, limit the scope of a height adjustment mechanism that may be included in any given embodiment of a knee crutch system. It is an advantage of some embodiments, however, that a height adjustment mechanism, such as exemplary mechanism 160, be comprised substantially within or proximate to post 145 so that a user's line of sight to prosthetic foot 155 is not obstructed. Regardless, one of ordinary skill in the art will recognize that any height adjustment mechanism operable to vary the height of a knee crutch system will be considered an equivalent that falls within the scope of the present disclosure.

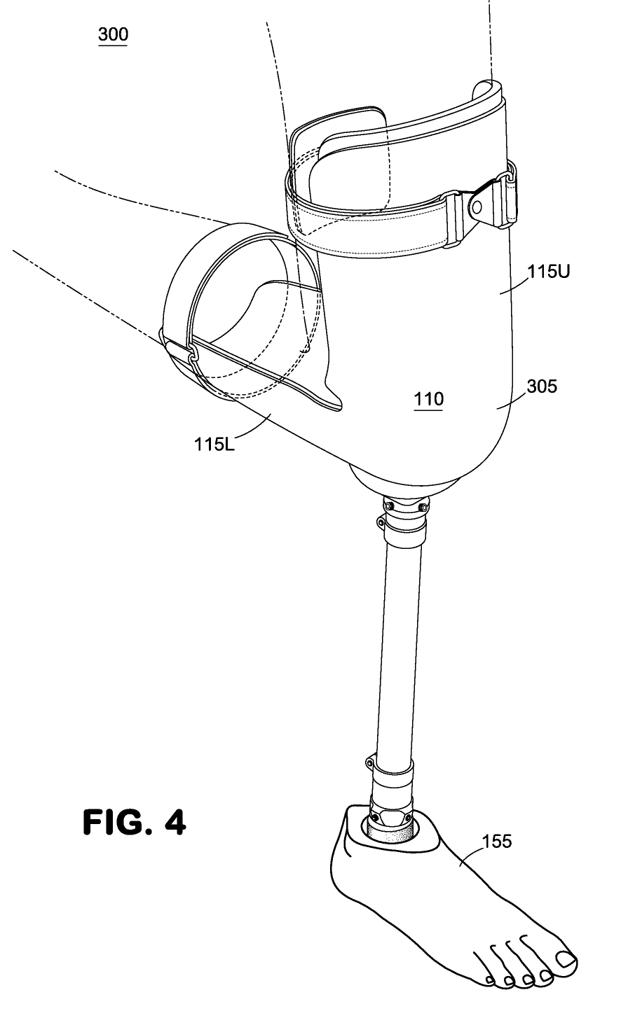

FIG. 4 is a perspective view of an exemplary knee crutch system 300 having an integral knee aspect 305. Unlike the embodiments depicted in FIGS. 1-3, the FIG. 4 embodiment 300 does not include a hinge 105 between the upper and lower leg portions 115U, 115L. As mentioned above, a hinge feature 105 is not required in all embodiments of a knee crutch system. Thus, the FIG. 4 embodiment 300 is offered to illustrate one such embodiment of a knee crutch system having an integral knee joint 305. One of ordinary skill in the art will recognize that an embodiment of a knee crutch system having an integral connection 305 between the upper leg portion 115U and the lower leg portion 115L of the cradle component 115 may provide some users with a more stable experience.

Turning to the FIG. 5 illustration, a top view is depicted of an exemplary knee crutch system in use by a user. From the top view, the lower right leg 405 of the user 410 can be seen supported by the lower leg portion 115L of the cradle 115. Moreover, prosthetic foot 155 can be seen extending in front of the user's person. That is, in some embodiments of a knee crutch system, when a user is in a standing position, the prosthetic foot 155 may be positioned substantially in parallel with the user's opposing foot 415. Advantageously, by providing for the prosthetic foot 155 to be positioned in correlation to a user's natural foot position, some knee crutch embodiments enable a user to more easily navigate and avoid extraneous obstacles. Also, because upper leg portion 115U may be configured to fit closely over the contour of the thigh of user 410, it is an advantage of some embodiments that the user's view to prosthetic foot 155 is not obscured.

Regarding prosthetic foot 155, it is offered for exemplary purposes only and is not intended to, nor does it, limit the scope of a knee crutch system such that an embodiment of a knee crutch system must include a prosthetic foot resembling prosthetic foot 155. It is envisioned that various embodiments of a knee crutch system may include any type of prosthetic foot or prosthetic foot system that would be suitable for a given user application. For example, various embodiments of a knee crutch system may include a prosthetic foot such as, but not limited to, a non-articulating flexible keel foot, a single or multi-axis articulating foot, a dynamic response foot, a pylon foot, a vertical shock pylon foot, solid ankle cushion heel foot, a Symes foot or the like. Moreover, because embodiments of a knee crutch system may accommodate various prosthetic feet or prosthetic feet systems known in the art, one of ordinary skill in the art will recognize that an advantage of many embodiments of a knee crutch system is that the shock absorbing qualities of such prosthetics may be leveraged. One of ordinary skill in the art will further recognize that an advantage of some embodiments of a knee crutch system which include certain prosthetic feet is that the footwear, i.e. “shoes,” of a user may be accommodated.

FIGS. 6A-6C collectively depict an exemplary knee crutch system in use through various stages of a walking motion. As described above, knee crutch systems are ambulatory. More specifically, embodiments of knee crutch systems provide support to a user's injured lower leg 405, safely positioning it such that it bears no weight other than its own, while enabling the user to remain mobile. Moreover, a user of a knee crutch system may enjoy mobility without obligating the use of his arms or hands to stabilize the ambulatory motion or otherwise support body weight.

In FIG. 6A, the user 410 is depicted in a stage of walking such that the prosthetic foot 155 of the exemplary knee crutch system is extended ahead of the user 410. The heel of the prosthetic foot is depicted as being in contact with a walking surface. The user's injured leg 405 is supported by lower leg portion 115L and the force of the user's weight is being transitioned from the user's “good” foot 415 to the forward positioned prosthetic foot 155.

In FIG. 6B, the user 410 has fully transitioned his weight onto prosthetic foot 155. Advantageously, the force from his body weight is transferred to the walking surface from his knee, down through post 145 and prosthetic foot 155. As the user's weight is fully carried by the exemplary knee crutch system, the user 410 may begin swinging his uninjured leg/foot 415 to a position forward of his body.

In FIG. 6C, the user 410 has positioned his uninjured leg/foot 415 forward of his body and has begun the transfer of weight from the exemplary knee crutch system to the uninjured leg/foot 415. Subsequently, the user 410 may continue the walking motion, repeating the process depicted in FIGS. 6A-6C, as would be understood by one of ordinary skill in the art.

Certain steps in the processes or process flows described in this specification naturally precede others for the invention to function as described. However, the invention is not limited to the order of the steps described if such order or sequence does not alter the functionality of the invention. That is, it is recognized that some steps may performed before, after, or parallel (substantially simultaneously with) other steps without departing from the scope and spirit of the invention. In some instances, certain steps may be omitted or not performed without departing from the invention. Further, words such as “thereafter”, “then”, “next”, “subsequently”, etc. are not intended to limit the order of the steps. These words are simply used to guide the reader through the description of the exemplary method.

The present knee crutch system has been described using detailed descriptions of embodiments thereof that are provided by way of example and are not intended to limit the scope of the system. The described embodiments comprise different features, not all of which are required in all embodiments of a knee crutch system. Some embodiments of a knee crutch system utilize only some of the features or possible combinations of the features. Variations of embodiments of a knee crutch system that are described and embodiments of a knee crutch system comprising different combinations of features noted in the described embodiments will occur to persons of ordinary skill in the art.

It will be appreciated by persons of ordinary skill in the art that a knee crutch system is not limited by what has been particularly shown and described herein above. Rather, the scope of a knee crutch system is defined by the claims that follow.

What is claimed is:

1. A device for supporting the mobility of a user who has an injury below the knee, the device comprising:
a cradle component configured substantially in a right angle such that an upper leg portion of the cradle component joins a lower leg portion of the cradle component to define a corner, wherein the cradle component is configured to interface to the user by receiving the upper leg of the user into the interior of the upper leg portion, the lower leg of the user into the interior of the lower leg portion and the knee of the user into the interior of the corner;
a shaft component having a proximate end in communication with the cradle component at the exterior of the cradle component corner and a distal end configured for communication with the ground, wherein the shaft is substantially aligned with the upper leg of the user; and
one or more securing components operable to secure the cradle component to the user;
wherein the upper leg portion of the cradle partially wraps around the upper leg of the user and the lower leg portion of the cradle supports the lower leg of the user in a position that is substantially parallel to the ground.

2. The device of claim 1, further comprising a hinging mechanism at the proximate end of the shaft component, wherein the hinging mechanism provides the user with the option of positioning the shaft component in a position that is substantially parallel to the lower leg portion of the cradle component.

3. The device of claim 1, wherein the shaft component further comprises a length adjustment mechanism.

4. The device of claim 1, wherein the shaft component further comprises a shock absorbing mechanism.

5. The device of claim 1, further comprising a prosthetic foot at the distal end of the shaft component.

6. The device of claim 5, wherein the prosthetic foot anatomically resembles a human foot.

7. The device of claim 5, further comprising a hinging mechanism between the distal end of the shaft component and the prosthetic foot.

8. The device of claim 5, wherein the prosthetic foot is comprised of a material selected from the group consisting of foam rubber, plastic, metal alloy and carbon-fiber composite.

9. The device of claim 5, wherein the prosthetic foot is configured to receive a shoe.

10. The device of claim 5, wherein the prosthetic foot is visible to the user beyond a plane defined by the exterior surface of the upper leg portion of the cradle.

11. A device for supporting the mobility of a user who has an injury below the knee, the device comprising:
a cradle component configured substantially in a right angle such that an upper leg portion of the cradle component operates in conjunction with a lower leg portion of the cradle component to define a corner, wherein the cradle component is configured to interface to the user by receiving the upper leg of the user into the interior of the upper leg portion, the lower leg of the user into the interior of the lower leg portion and the knee of the user into the interior of the corner;
a hinging mechanism at the point of communication between the upper leg and lower leg portions of the cradle, wherein the hinging mechanism is configured such that the position of the upper leg portion of the cradle may be translated relative to the lower leg portion of the cradle;
a shaft component having a proximate end in communication with the cradle component at the exterior of the cradle component corner and a distal end configured for communication with the ground, wherein the shaft is substantially aligned with the upper leg of the user; and
one or more securing components operable to secure the cradle component to the user;
wherein the upper leg portion of the cradle partially wraps around the upper leg of the user and the lower leg portion of the cradle supports the lower leg of the user in a position that is substantially parallel to the ground.

12. The device of claim 11, further comprising a hinging mechanism at the proximate end of the shaft component, wherein the hinging mechanism provides the user with the option of positioning the shaft component in a position that is substantially parallel to the lower leg portion of the cradle component.

13. The device of claim 11, wherein the shaft component further comprises a length adjustment mechanism.

14. The device of claim 11, wherein the shaft component further comprises a shock absorbing mechanism.

15. The device of claim 11, further comprising a prosthetic foot at the distal end of the shaft component.

16. The device of claim 15, wherein the prosthetic foot anatomically resembles a human foot.

17. The device of claim 15, further comprising a hinging mechanism between the distal end of the shaft component and the prosthetic foot.

18. The device of claim 15, wherein the prosthetic foot is comprised of a material selected from the group consisting of foam rubber, plastic, metal alloy and carbon-fiber composite.

19. The device of claim 15, wherein the prosthetic foot is configured to receive a shoe.

20. The device of claim 15, wherein the prosthetic foot is visible to the user beyond a plane defined by the exterior surface of the upper leg portion of the cradle.
